(19) United States
(12) Patent Application Publication
(10) Pub. No.: US 2025/0255495 A1
(43) Pub. Date: Aug. 14, 2025

(54) Physiological Strain Sensor

(71) Applicant: SIBEL HEALTH INC., Chicago, IL (US)

(72) Inventors: Ha Uk Chung, Niles, IL (US); Daniel Lopez, Niles, IL (US); Elena Kulikova, Niles, IL (US); Husein Gonzalez, Niles, IL (US)

(73) Assignee: SIBEL HEALTH INC., Chicago, IL (US)

(21) Appl. No.: 18/857,106

(22) PCT Filed: Apr. 12, 2023
(86) PCT No.: PCT/US2023/018387

§ 371 Date: Oct. 15, 2024

Related U.S. Application Data
(60) Provisional application No. 63/331,796, filed on Apr. 16, 2022.

(51) Int. Cl. A61B 5/0205; A61B 5/00; A61B 5/01; A61B 5/024; A61B 5/0245; A61B 5/11; A61B 5/282

(52) U.S. Cl. CPC A61B 5/02055; A61B 5/01; A61B 5/02416; A61B 5/0245; A61B 5/11; A61B 5/282; A61B 5/6823; A61B 5/6832; A61B 2560/0214; A61B 2560/0468; A61B 2562/0271; A61B 2562/12

(57) ABSTRACT

Wearable sensor apparatus and methods. The wearable sensor apparatus includes a body temperature sensor to be placed in the xiphoid process of a user, a heart rate sensor to be placed in any curvilinear surface of the upper trunk of the chest; a microcontroller unit (MCU) contained within the wearable sensor apparatus and in operable connectivity with the at least one sensor device and configured to receive the gathered data and process it onboard the wearable sensor; and a communication module in operable connectivity with the MCU and configured to wirelessly transmit at least some of the gathered data to an external receiver.

### CROSS REFERENCE TO RELATED APPLICATIONS

The present application claims the benefit of and priority to co-pending U.S. provisional application No. 63/331,796, filed on Apr. 16, 2022, the content of which is hereby incorporated by reference as if set forth in its entirety herein.

### TECHNICAL FIELD

Embodiments described herein generally relate to biosensor devices and methods and, more particularly but not exclusively, to a wireless sensor apparatus capable of recapitulating physical strain signals and communicating the same via a wireless network.

### BACKGROUND

It may be desirable to monitor a subject's physiological parameters in a variety of contexts. These contexts may include healthcare, athletics, consumer health, military, physical training, or contexts involving personnel in high-stress environments (e.g., firefighters, miners, industrial workers, etc.).

Existing technologies for monitoring physiological parameters involve a highly inconvenient setup. For example, measuring a subject's heart rate in situations in which the subject is sweating or moving can be difficult as the sensor device(s) may not remain secured to the subject. Additionally, these existing setups may be uncomfortable to the user.

There are also practical limitations to measuring temperature using rectal-, tympanic-, or esophageal-based thermometers. Recent technological developments include ingestible, telemetric thermometer pills. However, these ingestible technologies suffer from limited operating conditions, constraints due to telemetry protocol compatibility, and accuracy.

A need exists, therefore, for physiological measuring devices, systems, and methods that overcome the disadvantages associated with existing techniques.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

According to one aspect, embodiments relate to a wearable sensor apparatus. The apparatus includes a body temperature sensor to be placed in the xiphoid process of a user; a heart rate sensor to be placed in any curvilinear surface of the user's chest; a microcontroller unit (MCU) contained within the wearable sensor apparatus and in operable connectivity with the at least one sensor device and configured to receive the gathered data and process it onboard the wearable sensor apparatus; and a communication module in operable connectivity with the MCU and configured to wirelessly transmit at least some of the gathered data to an external receiver.

In some embodiments, the at least one sensor device includes a plurality of electrodes, and the heart rate data is derived from at least one of electrocardiogra data, seismocardiogram (SCG) data, or photoplethysmography (PPG) data.

In some embodiments, the heart rate sensor measures the heart rate during periods of high movement, high sweat conditions, or both.

In some embodiments, the MCU and the communication module are contained within a housing, and the at least one sensor device is removably attachable to the housing.

In some embodiments, the heart rate sensor includes two electrodes and an elastic portion to provide an adjustable spacing between the two electrodes.

In some embodiments, the MCU is further configured to process the heart rate data and the body temperature data to generate a value representative of physiological strain experienced by a user.

In some embodiments, the communication module wirelessly transmits at least some of the gathered data to the external receiver via a protocol selected from the group consisting of 4G, 5G, LTE, wireless fidelity (WiFi), and Bluetooth.

In some embodiments, the apparatus further includes an adhesive layer for removable attachment to a user to facilitate data gathering.

In some embodiments, the body temperature sensor gathers body temperature data and includes an analog front end (AFE), and at least one negative temperature coefficient (NTC) thermistor, wherein a change in resistance of the at least one NTC thermistor indicates a change in the body temperature of the user.

In some embodiments, the apparatus further includes a rechargeable power source.

In some embodiments, the communication module includes a near-field communication coil to enable wireless power transfer.

In some embodiments, the apparatus further includes a near-field communication tag configured to execute a cryptographic authentication procedure to authenticate the sensor apparatus prior to wirelessly transmitting the gathered data to the external receiver.

According to another aspect, embodiments relate to a method for manufacturing a wireless sensor apparatus. The method includes providing a housing; configuring the housing with at least one sensor device configured to gather heart rate data, body temperature data, or both; operably connecting the at least one sensor device with a microcontroller unit (MCU) configured to receive the gathered data and process it onboard the apparatus; and configuring the housing with a communication module to wirelessly transmit at least some of the gathered data to an external receiver.

In some embodiments, the at least one sensor device includes a plurality of electrodes, and the heart rate data is at least one of electrocardiogramata, seismocardiogram (SCG) data, and photoplethysmography (PPG) data.

In some embodiments, the at least one sensor device includes two electrodes, and the method further includes providing an elastic portion supporting the two electrodes to provide an adjustable spacing between the two electrodes.

In some embodiments, the MCU is further configured to process the heart rate data and the body temperature data to generate a value representative of physiological strain experienced by the user.

In some embodiments, the at least one sensor device gathers body temperature of the user and includes an analog front end (AFE), and at least one negative temperature coefficient (NTC) thermistor, wherein a change in resistance of the at least one NTC thermistors indicates a change in the body temperature of the user.

In some embodiments, the method further includes providing the housing with a rechargeable power source.

In some embodiments, the communication module includes a near-field communication coil to enable wireless power transfer, and encrypted communication with the external location.

In some embodiments, the method further includes providing a near-field communication tag configured to execute a cryptographic authentication procedure to authenticate the sensor apparatus prior to wirelessly transmitting the gathered data to the external receiver.

According to another aspect, embodiments relate to a wireless system. The wireless system includes a sensor apparatus as discussed above; and an external receiver at a location external to the sensor apparatus and configured to wirelessly receive gathered data from the sensor apparatus and wirelessly transmit a configuration parameter to the sensor apparatus.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a wearable sensor apparatus in accordance with one embodiment;

FIG. 2 illustrates a wearable sensor apparatus in accordance with another embodiment;

FIG. 3 illustrates a wearable sensor apparatus in accordance with another embodiment;

FIG. 4 illustrates an architecture diagram of a wearable sensor apparatus in accordance with one embodiment;

FIG. 5 illustrates an exploded view of an adhesive in accordance with one embodiment;

FIG. 6 illustrates the adhesive of FIG. 5 with respect to a wearable sensor apparatus and a user's skin in accordance with one embodiment;

FIG. 7 illustrates a workflow for transmitting physiological data in accordance with another embodiment;

FIG. 8 illustrates a workflow for receiving data from peripheral equipment in accordance with one embodiment;

FIG. 9 illustrates a workflow for transmitting physiological data in a healthcare environment using 2-factor authentication in accordance with one embodiment; and

FIG. 10 depicts a flowchart of a method for manufacturing a wireless sensor apparatus in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMS, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

The embodiments described herein provide novel sensor apparatuses, systems, and methods for obtaining physiological parameters of a user. The sensor apparatus may include a body temperature sensor and a heart rate sensor. The sensor apparatus in accordance with the embodiments herein may further include a microcontroller unit (MCU) that is contained within the wearable sensor apparatus and is in operable connectivity with the body temperature sensor and the heart rate sensor. The MCU may perform any processing steps onboard, and may wirelessly transmit at least some of the gathered data to an external receiver.

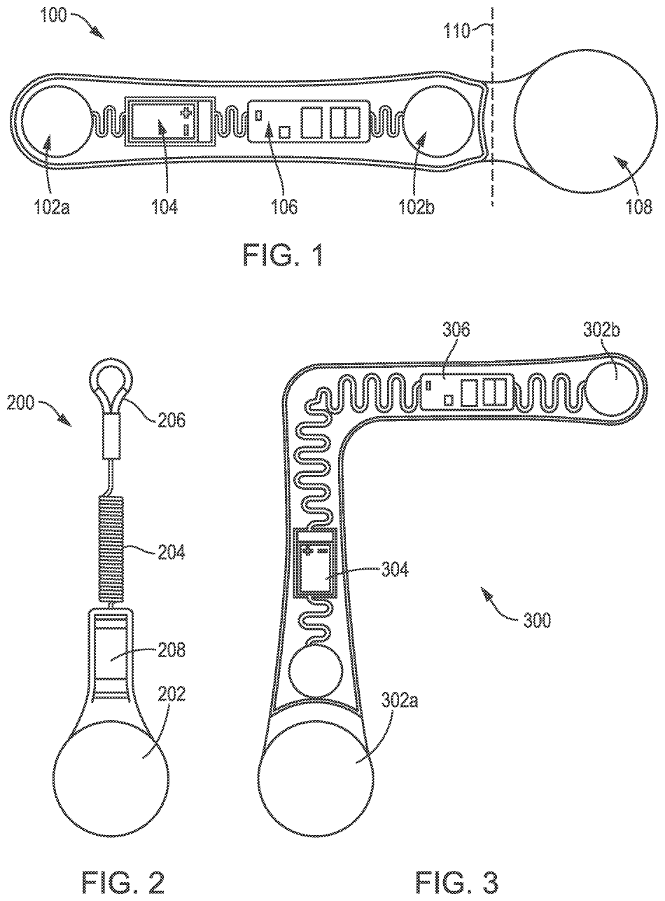

FIG. 1 illustrates a wearable sensor apparatus 100 in accordance with one embodiment. The sensor apparatus 100 may include, inter alia, electrodes 102a and 102b, a power source 104, and a circuit board 106. The sensor apparatus 100 may also include a temperature sensor 108. In this embodiment, the temperature sensor 108 may be detachable from the other components of the apparatus 100, as illustrated by the dashed line 110.

The power source 104 may be a rechargeable lithium polymer battery that can be charged via wireless power transfer by a transmitter. For example, the wearable sensor apparatus 100 may use a near-field communication (NFC) coil for wireless power transfer and for encrypted pairing and authentication with external devices or systems. The wearable sensor apparatus 100 may, in power transfer mode, receive transmitted wireless power from an NFC reader. The sensor 100 may then rectify and regulate the voltage to charge the battery through a power management integrated circuit (PMIC). The sensor apparatus 100 may thermally protect the power source 104 by a cap or housing portion made of a material with UL94 V-0 flame rating.

The body temperature sensor 108 may measure ambient, peripheral, and core body temperature. The body temperature sensor 108 may function completely detached from the other components of the sensor apparatus 100 as well. Thus, the embodiments herein may be modular in nature and allow for disposable, functional units to be attached to and removed from the sensor apparatus 100.

FIG. 2 illustrates a wearable sensor apparatus 200 in accordance with another embodiment. In this embodiment, one or more electrodes 202 may have a corded wire portion 204 connected to an ECG sensing unit (e.g., including another electrode) 206 to allow for adjustable ECG spacing. There is a positive correlation between the ECG amplitude and the spacing between electrodes. Additionally, extending this spacing further provides motion-proof QRS amplitude detection. The sensor apparatus 200 of FIG. 2 may also include any required power sources 208 or circuitry components.

FIG. 3 illustrates a wearable sensor apparatus 300 in accordance with another embodiment. The sensor apparatus 300 may include electrodes 302a and 302b, a power source 304, and any required circuitry components 306 (e.g., including a circuit board).

The sensor apparatuses such as those described in conjunction with FIGS. 1-3 may have a maximum thickness of 7.34 mm and maximum weight of 20 g. In addition, the sensor apparatus may be encapsulated within a highly flexible silicone material that is also thermally conductive. This form factor provides a mechanically soft and flexible structure that conformably mounts to the curvature of a user's body, such as to the curvature of a user's chest. The encapsulation may also provide protection against water ingress and dust, according to ingress protection (IP) 67 code. The thin, flexible, comfortable, and waterproof design provides superior wearing conditions for users such as safety workers who typically wear equipment such as BA and PPEs and operate in motion- or sweat-inducing conditions.

The sensor apparatuses described herein may have a certain aspect ratio with a maximum width of 12 mm for conformable placement of the sensor apparatus into a sternum location on a user. The low-durometer silicone encapsulation can be designed with a concave shape to accommodate users with general or atypical chest curvature.

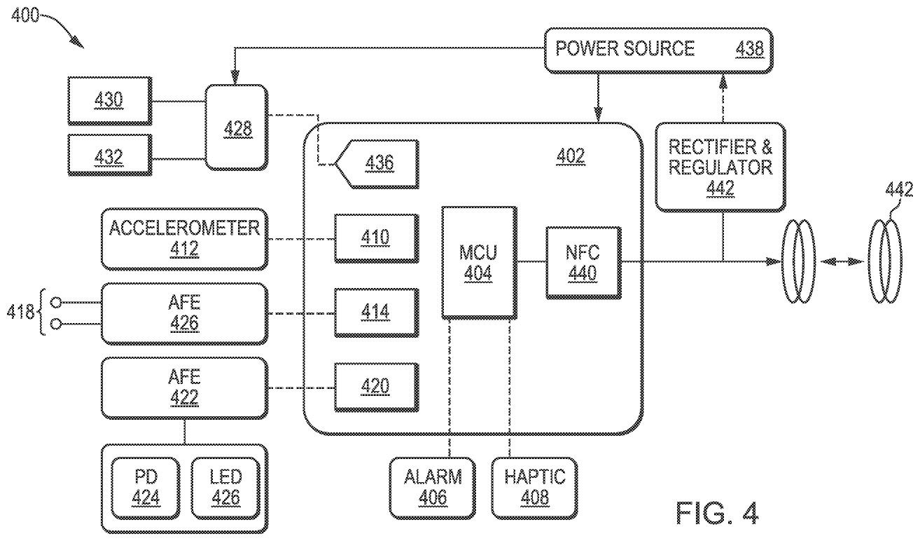

FIG. 4 illustrates an architecture diagram of a wearable sensor apparatus 400 in accordance with one embodiment. The apparatus 400 be similar to any of the sensor apparatuses of FIGS. 1-3 discussed above.

The apparatus 400 may include a Bluetooth-enabled system-on-a-chip (SoC) 402 for monitoring physiological parameters of a user. A microcontroller unit (MCU) 404 configures various sensing units and processes data gathered from the sensing units. The SoC 402 may then transmit gathered data via Bluetooth communications to one or more external devices, systems, storage locations, or the like.

Although the present application largely discusses Bluetooth communications, the embodiments herein may support other communication technologies or protocols. For example, the sensor apparatus 400 may support protocols such as 4G, 5G, LTE, ZigBee, Bluetooth Low Energy (BLE), and Wi-Fi. This list is exemplary and other communication protocols whether available now or invented hereafter may be used in conjunction with the embodiments described herein.

The apparatus 400 may also receive data from other Bluetooth-enabled devices to trigger alarming events through visual means, auditory means, haptic-based means, or some combination thereof. For example, the MCU 404 may be in further communication with an alarm module 406 and a haptic module 408. Accordingly, the apparatus 400 may issue alerts to a user, wherein the alerts may be associated with particular instructions for the user such as to cease physical activity.

The MCU 404 may include various combinations of resistors, transistors, and diodes to form any number of logic gates to execute appropriate programs. The MCU 404 may store any combination of types of memory such as RAM memory configurations. The memory may include non-volatile memory such as flash memory, EPROM, EEPROM, ROM, and PROM, or volatile memory such as static or dynamic RAM, as discussed above. The exact configuration/type of onboard memory may of course vary as long as instructions for analyzing data from the various other components of the apparatus 100 may be performed by the MCU 404.

The SoC 402 may communicate with various sensor components through the serial peripheral interface (SPI) protocol. For example, an SPI 410 may allow the SoC 402 to configure and communicate with an accelerometer 412, SPI 414 may allow the SoC 402 to configure and communicate with an analog front end (AFE) 416 for receiving data from one or more electrodes 418, and SPI 420 may allow the SoC 402 to configure and communicate with an AFE for measuring a PPG signal.

The SPI 410 may configure the accelerometer(s) 412, sampling at 1 kHz, for example. The accelerometer 412 may be a 3-axis accelerometer configured to measure acceleration of a user in different axes.

The SPI 414 may configure the ECG AFE 416, sampling at 512 Hz, for example. The ECG AFE 416 may receive data from one or more electrodes 418 placed on a user. For example, two dry electrodes 418 may be placed on a user's chest. These electrodes may be coated by gold (Au, 0.05 μm) for durable operation, even in the presence of sweating or user motion. In some embodiments, such as those of FIG. 1B and FIG. 1C, the electrodes 418 may have adjustable spacing through a stretchable, umbilical connection mechanism.

The SPI 420 may configure the PPG AFE 422, sampling at 128 Hz, for example. The PPG AFE 422 may include or otherwise be in communication with a broadband photodiode 424 and one or more light emitting diodes (LEDs) 426. For example, the LEDs 426 may include LEDs in wavelengths of red (660 nm), green (526 nm), and infra-red (IR, 950 nm). The PPG AFE 422 may sample signals collected through each of these LEDs, for example.

In some embodiments, a single MCU such as the MCU 404 may configure the ECG AFE 416 and PPG AFE 422. For example, the MCU 404 may configure the various AFEs to provide a time-synchronized time delay through a high frequency clock (HFCLK) connected to the MCU 404. The so-called pulse arrival time (PAT) may be calculated from the peak-to-foot timing between the ECG and PPG waveforms. The PAT calculation provides highly accurate timing information to obtain the surrogate measurement of blood pressure without, for example, requiring large and unwieldy blood pressure monitors. Additionally, these techniques provide highly-controlled timing accuracy with power-efficient operation compared to other techniques for measuring PAT that employ a wireless sensor network and require high power demands.

The SoC 402 may further include or otherwise be in communication with a temperature sensing unit 428. The temperature sensing unit 428 may include a negative temperature coefficient (NTC) thermistor 430 for measuring ambient temperature, and an NTC thermistor 432 for measuring peripheral temperature. The resistance of the NTC thermistors 430 and 432 changes inversely proportional to temperature change. The resistance is then processed through a resistor ladder circuit with a shunt resistor to limit the amount of current flowing through the NTC thermistors 430 and 432 to avoid or at least minimize self-heating. The temperature sensing unit 428 may further include a filter such as a passive 100 Hz low pass filter for filtering the output before transmitting the processed temperature data to an analog-to-digital converter (ADC) 436. The ADC 436 may be a successive approximation ADC with up to a 14-bit resolution, for example.

The MCU 404 may calculate a parameter referred to as a physiological strain index (PSI), which is an estimate of a combination of heat stress, heat stroke, and heat exhaustion. PSI may be derived by any function that includes a heart rate input and a body temperature input. Ambient conditions, such as external temperature and humidity, may also be a factor in determining PSI.

PSI may be estimated according to the following formula:

$$PSI = 5 ( {T}_{f} - {T}_{0} ) ( 39.5 - {T}_{0} ) - 1 + 5 ( {HR}_{f} - {HR}_{0} ) ( 1 \; 80 - {HR}_{0} ) - 1$$

where T_{f} and HR_{f} are measurements of temperature and heart rate, respectively, at the moment of interest, while T₀ and HR₀ are the measurements of temperature and heart rate, respectively, at the initial measurement.

The power source 438 may be a rechargeable lithium polymer battery that can be charged via wireless power transfer by a transmitter. For example, the wearable sensor apparatus 400 may use a near-field communication (NFC) coil 440 for wireless power transfer and for encrypted pairing and authentication. The wearable sensor apparatus 400 may, in a power transfer mode, receive transmitted wireless power at 13.56 MHz from an NFC reader 442. The sensor then uses a voltage rectifier & regulator 442 to rectify and regulate the voltage to charge the power source 436 through a power management integrated circuit (PMIC). The sensor 400 or housing thereof may thermally protect the power source 436 with full coverage by a cap made of a material with a UL.94 flame rating.

The NFC coil 440 may execute one or more cryptographic algorithms including, but not limited to AES-128, TDES, RSA, and ECC. Some embodiments may use two-factor authentication, such as using a mobile device to cross-validate between the signature data of the sensor and a public key that can be obtained from either cloud or a local storage of a user device, discussed below.

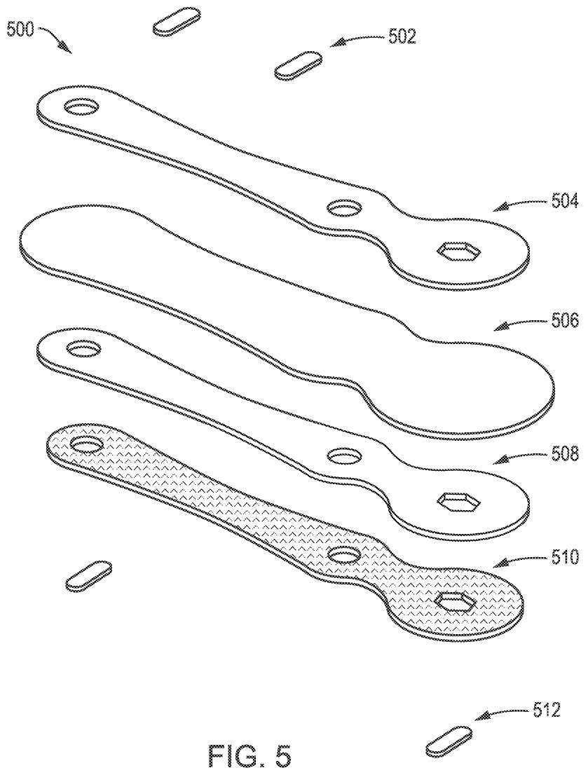

FIG. 5 illustrates an exploded view of an adhesive 500 in accordance with one embodiment. The adhesive 500 may be positioned such that the bottom of the adhesive 500 contacts the user (e.g., the user's skin), and the top layer of the adhesive 500 contacts a sensor apparatus such as the wearable sensor apparatus of FIGS. 1-4.

The adhesive 500 may include multiple layers, including a layer of conductive film 502 to contact the sensor apparatus, a first silicone gel layer 504, a fluid-resistant foam layer 506, a moisture-wicking fabric layer 508, a second silicone gel layer 510 with perforation, and a layer of spaced conductive films 512

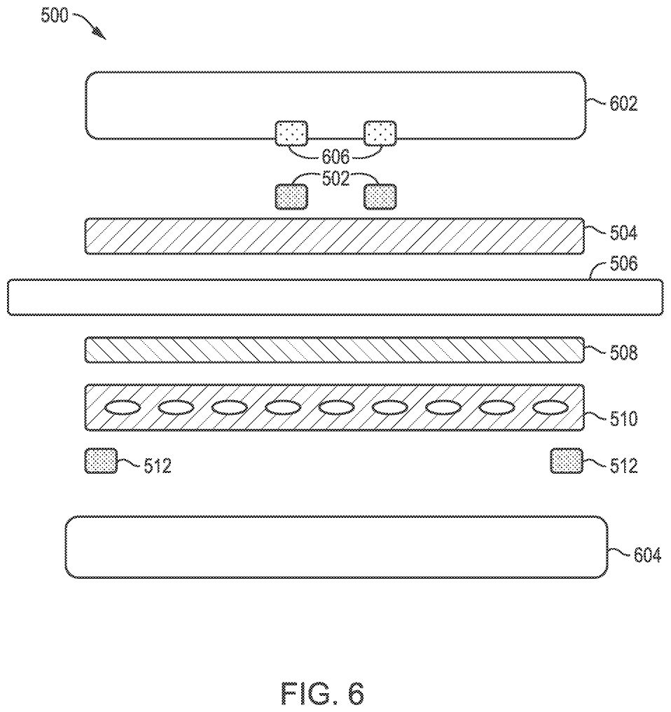

FIG. 6 illustrates the adhesive 500 of FIG. 5 with respect to an ECG sensor 602 and a user's skin 604 in accordance with one embodiment. As seen in FIG. 6, the spaced conductive films 502 separate the dry electrodes 606 of the ECG sensor 602.

The silicone gel layer 504 may provide adhesion to the sensor apparatus through siloxane bonding. The second silicone gel layer 510 provides a channel for moisture or sweat to dissipate toward the moisture-wicking fabric layer 508 by capillary action. The moisture-wicking fabric layer 508 may manage or absorb the sweat until reaching a saturation point.

The fluid-resistant foam layer 506 surrounds the overall adhesive 500 and provides an additional area of contact between the sensor apparatus and skin 604. For example, the fluid-resistant foam layer 506 may increase this contact area by approximately 20%. The fluid-resistant foam layer 506 provides moisture-proof adhesion to the skin 604 around the contact area, and provides a way for moisture trapped inside the moisture-wicking fabric layer 508 to exit when the rate of natural evaporation is exceeded. The adhesive 500 can therefore resist environmental moisture and moisture created at the interface of the skin 604 and the adhesive 500.

There may be some loosening of the adhesive 500 due to moisture generation during operation. This loosening may facilitate the intentional removal of the sensor apparatus at the end of usage and without resulting in skin injuries as damages.

In some embodiments, the adhesive 500 may have the aforementioned layers 502-512 where each side of 502 and 512 are internally and electrically connected (not shown in the FIG. 6) to extend the electrode spacing beyond the distance defined by the dry electrodes 606. However, in these embodiments, the second silicone gel layer 510 may be replaced by a gentle hydrogel material, such as for accommodating users having fragile skin. In these embodiments, the adhesive 500 may provide a gentle attachment to, e.g., underdeveloped skin of neonates in early gestational age while maintaining good adhesion for several days through the same moisture management methodologies described above.

In some embodiments, the adhesive 500 also provides a way to adjust the spacing between electrodes. This allows for improved ECG measurements, as the ECG locations can be placed in the most optimal position and to support different lead configurations.

The wearable sensor apparatus in accordance with embodiments herein may transmit physiological data, including PSI values, over a wireless network to one or more platforms for analysis. For example, the wearable sensor apparatus may transmit processed data to at least one of a patient database, a cloud-based server, a mobile device, or healthcare monitoring equipment. These systems or devices may notify a medical practitioner or otherwise a caregiver when the sensor apparatus detects an alarming or potentially-alarming physiological condition.

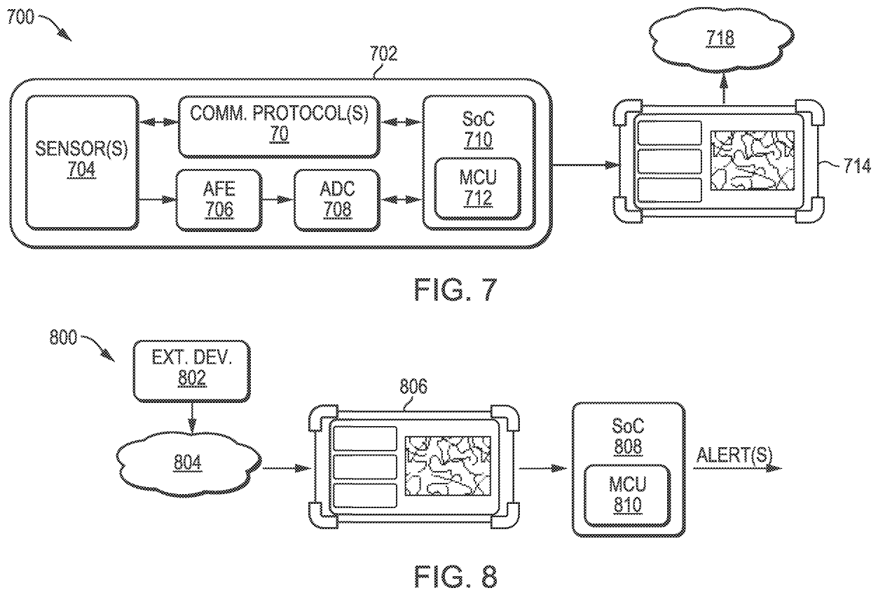

FIG. 7 illustrates a workflow 700 for transmitting physiological data using a sensor apparatus 702 in accordance with one embodiment. The sensor apparatus 702 may be similar to the sensor apparatus of any one or more of FIGS. 1-4. The workflow 700 may involve sending physiological data to a cloud-based environment, for example.

In operation, one or more sensors 704 such as an accelerometer, electrode(s), microphone, photoplethysmography sensor, or some combination thereof, communicate an analog signal to a corresponding analog front end (AFE) 706. The AFE 706 may forward the analog signal(s) to an ADC 708 for converting the analog signal(s) to a digital signal(s).

The converted digital signals may then be passed to a SoC 710 such as the SoC 402 of FIG. 4. An MCU 712 such as the MCU 404 may perform required processing or configuring procedures on the received data and transmit the processed data to a user device 714. The user device 714 may be, for example and without limitation, a smartphone, tablet, PC, smartwatch, or the like. The SoC 710 may also communicate any communication protocols or configuration parameters 716 to the sensor(s) 704. These may refer to or include serial communication protocols, for example.

The device 714 may display physiological parameters of multiple users or subjects and other key metrics from various equipment attached to or otherwise monitoring the user(s). In healthcare embodiments, the device 714 may also gather or display metrics such as oxygen level in a self-contained breathing apparatus (SCBA) or the output of gas detection equipment. These metrics may then be used to activate alarm events through the aforementioned visual, physical, and/or haptic based alarm methods. Additionally or alternatively, these metrics may be stored in or communicated over one or more cloud-based networks 718.

FIG. 8 illustrates a workflow 800 for receiving data from peripheral equipment in accordance with one embodiment. An external device such as SCBA, PPE, gas detection equipment or the like may transmit metrics regarding the status of a user over a network 804. The network 804 may be a cloud-based network, for example. A monitor 806 may download the metrics from the network 804 and may visually present data associated with the metrics.

The monitor 806 may also forward any appropriate metrics to a SoC 808 executing an MCU 810. The SoC 808 and the MCU 810 may be similar to the SoC 402 and the MCU 404, respectively, of FIG. 4. The SoC 808 may then issue one or more alerts to the user instructing them to, for example, cease physical activity or move to a safer environment. Additionally or alternatively, the alerts may be communicated to healthcare personnel.

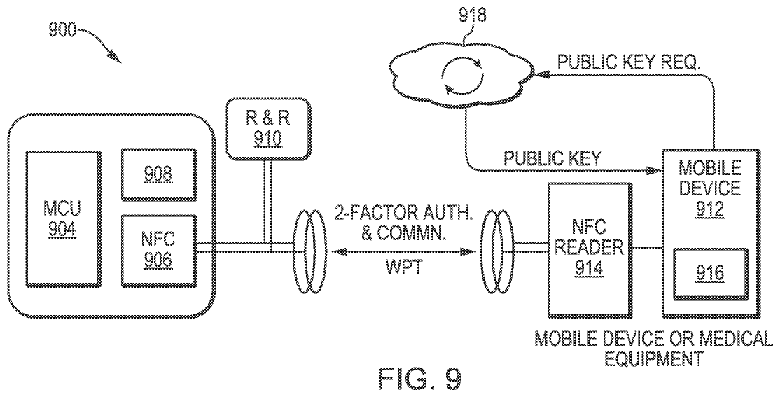

FIG. 9 illustrates a workflow 900 for transmitting physiological data in a healthcare environment in accordance with one embodiment. As in previous figures, a SoC 902 of a sensor apparatus may execute or otherwise include an MCU 904, an NFC 906, and memory 908. The SoC 902 may also include or be in communication with a voltage regulator and rectifier 910 for e.g., rectifying and regulating voltage to charge a power source (not shown in FIG. 9).

The SoC 902 may transmit physiological data with encryption through the NFC 906 to a mobile device 912 or medical equipment with a compatible NFC reader 914. The mobile device 912 or other type of medical equipment may execute instructions stored on memory 916 to perform 2-factor authentication. For example, the mobile device 912 may generate a public key request to a cloud-based or local storage 918 of the mobile device 912. The mobile device 912 or other type of medical equipment may receive the public key and validate the signature data of the NFC 906 and enable communication, wireless power transfer, or both.

In some embodiments, the sensor can transmit physiological data obtained from the sensor apparatus with encryption through an NFC to a mobile device with a compatible NFC reader and coil within the effective range. For example, the mobile device may be connected to an NFC embedded in a firefighter's equipment that is in proximity to a sensor apparatus mounted on the firefighter's chest for bi-directional communication.

In a medical setting, one embodiment may involve a coil embedded in a bed frame or incubator that is connected to a mobile device or medical equipment such as a medical monitor to stream data from a sensor apparatus on a patient's chest. In some embodiments, battery-free operation is enabled through wireless power transfer using the NFC reader on the mobile device or equipment on the sensor, with the sensor apparatus receiving transmitted power through its NFC and using it to power its on-board electronics.

In some embodiments, wireless power transfer from an NFC reader embedded in a uniform or clothing extends the duration of sensor apparatus operation by power scavenging. The sensor apparatus can have a continuous power source either with or without having an on-board battery if there is power remaining in the embedded device. Such device can be a mobile device, SCBA, PPE, or others.

In some embodiments, the sensor apparatus may provide a non-contact authentication method through quick response (QR) code scanning. The code can be directly printed on a surface of the sensor apparatus through, e.g., a laser engraving using high resolution lasers like ultraviolet lasers or fiber lasers. In some embodiments, the QR code is made by a standard printer and then attached to a side of the PCB using a tape or other adhesive. This code can be read by an external QR code scanner through, e.g., a transparent window in the encapsulation. Other codes may be used as well

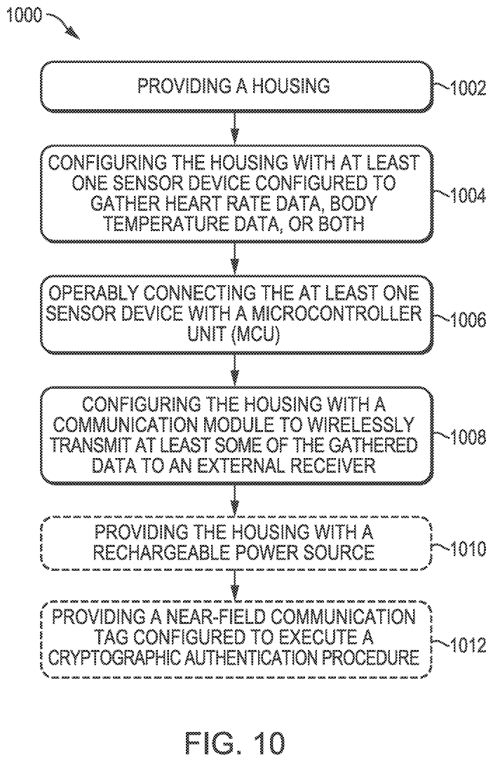

FIG. 10 depicts a flowchart of a method 1000 for manufacturing a wireless sensor apparatus in accordance with one embodiment. The wireless sensor apparatus referred to in conjunction with method 1000 may be similar to the sensor apparatus of FIGS. 1-4, for example.

Step 1002 involves providing a housing. The housing may be made of any suitable material that can conform to the contours and movement of a user's body and can protect the interior components of the housing.

Step 1004 involves configuring the housing with at least one sensor device configured to gather heart rate data, body temperature data, or both. For example, as in FIGS. 1-4, the wireless sensor apparatus may include one or more electrodes to gather ECG data. The sensor apparatus may also include a temperature sensor to gather temperature of a user, ambient temperature, etc. The temperature sensor may be placed the xiphoid process of a user.

Step 1006 involves operably connecting the at least one sensor device with a microcontroller unit (MCU) configured to receive the gathered data and process it onboard the apparatus. The MCU may be similar to the MCU 404 of FIG. 4, for example.

Step 1008 involves configuring the housing with a communication module to wirelessly transmit at least some of the gathered data to an external receiver. The communication module may include a near-field communication (NFC) coil to enable wireless power transfer, for example.

Step 1010 involves providing the housing with a rechargeable power source. In some embodiments, the power source may be a rechargeable lithium polymer battery that can be charged via wireless power transfer by a transmitter.

Step 1012 involves providing a near-field communication tag configured to execute a cryptographic authentication procedure to authenticate the sensor apparatus prior to wirelessly transmitting the gathered data to the external receiver. In some embodiments, the external receiver may be associated with a healthcare monitoring system. In some embodiments, the external receiver may be associated with a worker safety monitoring system for monitoring workers in high-stress and potentially dangerous environments.

Additionally or alternatively, the user device may refer to a monitor in a healthcare setting used to monitor a user (e.g., a patient). Accordingly, medical personnel or caregivers may monitor a user's health. The sensor apparatus and the monitoring technology described herein may be integrated into any medical equipment including, but not limited to, a ventilator, an incubator, an infusion pump, a hemodialysis machine, a radiological machine for imaging, or the like. This list is only exemplary and other types of monitoring or peripheral equipment may be used in conjunction with the sensor apparatus described herein.

The features of the described embodiments may be implemented in a variety of applications. In the case of worker monitoring and safety, the combination of parameters measured by the sensor apparatus (i.e., heart rate and core body temperature) provides the ability to track physiological strain. Physiological strain may be determined via a variety of equations but typically includes an input of at least heart rate and temperature from the body. For example, the PSI discussed previously may indicate whether an individual is at risk of heart strain, heat stress, heart stroke, or otherwise at risk of a condition requiring the user to cease physical activity or move to a cooler environment.

The provided alerts can indicate when a user should rest before harm occurs or may occur. The disclosed embodiments can be used in fields in which high physical activity and high ambient temperatures occur. These fields may include firefighting, mine working, construction work, and agricultural work.

In addition to or in lieu of worker monitoring, the disclosed embodiments can be used in a variety of other use cases. This includes athletics and sports where physiological exertion occurs.

In healthcare-related settings, the ability to continuously track physiological parameters such as heart rate, respiratory rate, and temperature-related parameters has numerous applications. These applications can include those in both critical care and ambulatory monitoring.

Specifically, tracking an infection status with features of the disclosed embodiments would be particularly important. The ability to continuously track parameters such as heart rate, respiratory rate, and temperature is highly relevant for monitoring various stages of the infection (e.g., onset of infection, improvement of infection, or worsening of infection). For patients at high risk, such as those with immunosuppression, malignancies, or recent bone marrow transplants, the disclosed embodiments can be used as a monitoring, tracking, and alerting tool. As discussed above the embodiments herein can send alert(s) to a wide variety of systems and devices.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A wearable sensor apparatus comprising:
a body temperature sensor to be placed in a xiphoid process of a user;
a heart rate sensor to be placed in any curvilinear surface of the user's chest;
a microcontroller unit (MCU) contained within the wearable sensor apparatus and in operable connectivity with at least one of the body temperature sensor and the heart rate sensor to receive gathered data from the at least one of the body temperature sensor and the heart rate sensor and to the gathered data onboard the wearable sensor apparatus; and
a communication module in operable connectivity with the MCU and configured to wirelessly transmit at least some of the gathered data to an external receiver and to wirelessly receive data from an external device.

2. The sensor apparatus of claim 1 wherein the heart rate sensor includes a plurality of electrodes, and the gathered data is derived from at least one of electrocardiogramata, seismocardiogram (SCG) data, or photoplethysmography (PPG) data.

3. The sensor apparatus of claim 1 wherein the heart rate sensor measures the heart rate during periods of high movement, high sweat conditions, or both.

4. The sensor apparatus of claim 1 wherein the MCU and the communication module are contained within a housing, and the at least one of the temperature sensor and the heart rate sensor is removably attachable to the housing.

5. The sensor apparatus of claim 1 wherein the heart rate sensor includes two electrodes and an elastic portion to provide an adjustable spacing between the two electrodes.

6. The sensor apparatus of claim 1 wherein the MCU is further configured to process the gathered data to generate a value representative of physiological strain experienced by a user.

7. The sensor apparatus of claim 1 wherein the communication module wirelessly transmits at least some of the gathered data to the external receiver via a protocol selected from the group consisting of a broadband wireless protocol, a local area network wireless protocol, and a near-field wireless protocol.

8. The sensor apparatus of claim 1 further comprising an adhesive layer for removable attachment to a user to facilitate data gathering.

9. The sensor apparatus of claim 1 wherein the body temperature sensor gathers body temperature data and includes:
an analog front end (AFE), and
at least one negative temperature coefficient (NTC) thermistor,
wherein a change in resistance of the at least one NTC thermistor indicates a change in the body temperature of the user.

10. The sensor apparatus of claim 1 further comprising a rechargeable power source.

11. The sensor apparatus of claim 1 wherein the communication module includes a near-field communication coil to enable wireless power transfer.

12. The sensor apparatus of claim 1 further comprising a near-field communication tag configured to execute a cryptographic authentication procedure to authenticate the sensor apparatus prior to wirelessly transmitting the gathered data to the external receiver.

13. A method for manufacturing a wireless sensor apparatus, the method comprising:
providing a housing;
configuring the housing with at least one sensor device configured to gather heart rate data, body temperature data, or both;
operably connecting the at least one sensor device with a microcontroller unit (MCU) configured to receive the gathered data and process it onboard the apparatus; and
configuring the housing with a communication module to wirelessly transmit at least some of the gathered data to an external receiver and to wirelessly receive data from an external device.

14. The method of claim 13 wherein the at least one sensor device includes a plurality of electrodes, and the heart rate data is at least one of electrocardiograma, seismocardiogram (SCG) data, and photoplethysmography (PPG) data.

15. The method of claim 13 wherein the at least one sensor device includes two electrodes, and the method further includes providing an elastic portion supporting the two electrodes to provide an adjustable spacing between the two electrodes.

16. The method of claim 13 wherein the MCU is further configured to process the heart rate data and the body temperature data to generate a value representative of physiological strain experienced by the user.

17. The method of claim 13 wherein the at least one sensor device gathers body temperature data of the user and includes:
an analog front end (AFE), and
at least one negative temperature coefficient (NTC) thermistor,
wherein a change in resistance of the at least one NTC thermistors indicates a change in the body temperature of the user.

18. The method of claim 13 further comprising providing the housing with a rechargeable power source.

19. The method of claim 13 wherein the communication module includes a near-field communication coil to enable:
wireless power transfer, and
encrypted communication with the external location.

20. The method of claim 13 further comprising providing a near-field communication tag configured to execute a cryptographic authentication procedure to authenticate the sensor apparatus prior to wirelessly transmitting the gathered data to the external receiver.

21. A wireless monitoring system comprising:
the sensor apparatus of claim 1; and
an external device including a receiver and a transmitter at a location external to the sensor apparatus of claim 1 and configured to:
wirelessly receive the gathered data from the sensor apparatus of claim 1, and
wirelessly transmit a configuration parameter to the sensor apparatus of claim 1.
